(19) United States
(12) Patent Application Publication
(10) Pub. No.: US 2011/0262897 A1
(43) Pub. Date: Oct. 27, 2011

(54) IMAGING TECHNIQUES USING A TRIDENTATE LIGAND

(75) Inventors: J. A. Gareth Williams, Durham (GB); Julia A. Weinstein, Sheffield (GB); Stanley W. Botchway, Oxon (GB); John Haycock, Sheffield (GB)

(73) Assignee: SCIENCE AND TECHNOLOGY FACILITIES COUNCIL, Oxfordshire (GB)

(21) Appl. No.: 13/122,361

(22) PCT Filed: Oct. 5, 2009
(86) PCT No.: PCT/GB2009/002369

§ 371 Date: Jul. 15, 2011

(30) Foreign Application Priority Data
Oct. 3, 2008 (GB) 0818151.3

(51) Int. Cl. C12Q 1/70; G01N 33/53; G01N 33/567; C07H 21/04; C12N 7/00; C07K 14/00; C07K 16/00; C07H 21/02; C12Q 1/68; C12Q 1/02

(52) U.S. Cl. 435/5; 435/6.1; 435/7.1; 435/7.2; 435/29; 435/235.1; 530/350; 530/391.3; 536/23.1

(57) ABSTRACT

The present invention relates to microscopy and, in particular, Time-resolved Emission Imaging Microscopy (TREM). The Invention relates to the use of a transition metal complex having a tridentate ligand in an imaging technique. The transition metal is preferably platinum.

The present invention relates generally to the field of microscopy and, in particular, Time-Resolved Emission Imaging Microscopy (TREM) for non-invasive imaging and mapping of live cells.

Understanding chemical processes that underpin biological systems relies on technological and conceptual developments which permit non-invasive monitoring of biological processes in real time. Fluorescence microscopy is one of the most widely used tools in the biological sciences, benefiting from exquisite sensitivity, excellent spatial resolution, and unparalleled temporal resolution. It permits a breadth of investigations from interactions of single molecules to whole organism studies and relies on light emission from either endogenous fluorophores or external fluorescent probes to monitor the local environment and changes therein. Of the three fundamental emission parameters—intensity, wavelength, and lifetime—the vast majority of fluorescence imaging studies to date have been based on spatial variation of emission intensity or on wavelength changes. In contrast, lifetime-based approaches remain severely underexplored. Yet lifetime-based sensing and imaging is a powerful complement to the usual methods. A major benefit is that emission lifetimes are normally independent of concentration and can be calibrated absolutely, unlike intensity measurements which are subject to error from fluctuations in the efficiency of delivery and detection of light.

Recent advances in fluorescence lifetime detection techniques have made fluorescence lifetime imaging microscopy (FLIM) a reality. To date, FLIM has been based primarily on endogenous molecules, such as the aromatic amino acid tryptophan, and on fluorescent dyes including GFP-tagged proteins and fluorescein derivatives, all of which have lifetimes of a few nanoseconds. The short lifetimes necessitate sub-nanosecond light sources and fast detectors, since: (i) relatively small changes within the sub-nanosecond timescale need to be resolved; and (ii) it becomes essential to distinguish the fluorescence of the agent from autofluorescence which emanates from natural biological chromophores also on the nanosecond timescale.

A significant step forward would be the development of time-resolved emission imaging microscopy (TREM) which uses longer timescales—hundreds of nanoseconds to microseconds. Long timescales offer improved discrimination through much larger changes in lifetime and by allowing time-gated experiments to distinguish from short-lived autofluorescence. TREM does not need fast excitation or detection methods; on the contrary, it can be performed with nanosecond lasers and slower gated detectors. To date, the limiting factor that has inhibited the development of practicable TREM has been the chemistry rather than the optical technology. The fundamental constraint is the lack of cell-permeable, non-toxic luminescent probes with lifetimes in excess of about 100 ns in aerated aqueous media at room temperature or above. Most phosphorescent organic molecules cannot be used under such conditions due to efficient quenching of their naturally long-lived triplet states.

Certain transition metal complexes can emit efficiently with lifetimes in excess of 100 ns from triplet excited states, since the formally spin-forbidden S₀←T₁ transition is promoted by the spin-orbit coupling associated with the heavy metal atom. Steady-state cell images have been obtained recently using ruthenium(II) and rhenium(I) complexes. The temporal dimension has yet to be explored. Successful time-gated cell imaging with charge-neutral platinum porphyrins has been reported, however, the long excited state lifetimes of ˜100 μs lead to severe oxygen quenching and consequent cytotoxicity.

The present invention aims to address at least some of the problems associated with the prior art.

Accordingly, the present invention provides for the use of a transition metal complex having a tridentate ligand in an imaging technique.

The present invention will now be further described. In the following passages different aspects/embodiments of the invention are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The transition metal complex may be used as a labelling agent, for example as a labelling agent in a cell.

The transition metal complex may be introduced into a cell in vitro or in vivo.

The transition metal complex may be pre-bound to a chemical species that is introduced into a cell. The chemical species may be a biomolecule. The chemical species may comprise, for example, a protein, an antibody, DNA, RNA, an antigen or a virus.

The transition metal complex may bind to active sites within a cell to label at least a portion of the cellular structure. Such active sites include, for example, nucleic acid active sites within a cell, preferably RNA and/or DNA active sites. The active sites may be within the nucleus or nucleoli of a cell, or within other sub-cellular structures or membranes. The complex does not have to be used in relation to cells alone. It can be used to bind to extra-cellular active sites, such as nucleic acids, RNA and/or DNA extracted from cells or synthesised.

The transition metal complex preferably has a quantum yield of light emission of 0.6 or greater, more preferably 0.65 or greater and most preferably 0.7 or greater.

The transition metal preferably has a square planar coordination.

Tridentate ligand preferably means that the ligand is coordinated to the transition metal at three coordination points.

The transition metal is preferably platinum.

The platinum complex is preferably a Pt^{(II)} complex.

The platinum complex is preferably charge neutral.

Advantageously, the platinum complex is of the formula Pt[L]X, wherein L is a tridentate ligand and X is a monodentate ligand. The tridentate ligand (L) is preferably a cyclometalating ligand. The tridentate ligand (L) preferably coordinates to the transition metal via N̂ĈN coordination points.

In a particularly preferred embodiment, the tridentate ligand (L) is 1,3-di(2-pyridyl)benzene or a derivative thereof. The tridentate ligand (L) is preferably a 1,3-di(2-pyridyl)benzene derivative substituted at the 4′ position.

The complex may be synthesised using known techniques, for example, as described in Williams J A G, Beeby A, Davies E S, Weinstein J A, Wilson C (2003) An alternative route to highly luminescent platinum(II) complexes: cyclometalation with N̂ĈN-coordinating dipyridylbenzene ligands. Inorg Chem 42:8609-8611.

Whatever the tridentate ligand (L), it is preferably substituted (most preferably at the 4′ position) with a bio-targeting functionality or a linking group suitable for reactively attaching the derivative to a bio-targeting functionality. The linking group may be an amide group or an ester group, for example.

X in the complex is preferably a monodentate pi donor ligand.

In a particularly preferred embodiment, the transition metal complex has the formula:

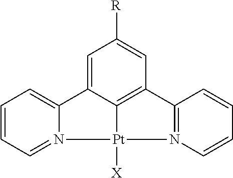

wherein R is —H, —C(O)OCH₃, —CH₃, or —C₆H₄—N(CH₃)₂; and wherein X is a monodentate ligand.

X may be, for example, Cl, Br, F or OH. Cl is preferred.

As noted above, the complex may be synthesized by conventional techniques such as described in Williams J A G, Beeby A, Davies E S, Weinstein J A, Wilson C (2003) An alternative route to highly luminescent platinum(II) complexes: cyclometalation with N̂ĈN-coordinating dipyridylbenzene ligands. Inorg Chem 42:8609-8611.

The present invention relates to an imaging technique, for example microscopy.

Examples include one- or multi-photon imaging and fluorescence and emission microscopy. In particular, the imaging technique may comprise fluorescence lifetime imaging microscopy (FLIM), time-resolved emission imaging microscopy (TREM), multi-photon excitation (MPE), two-photon excitation microscopy (TPE), Förster resonance energy transfer microscopy (FRET), epi-fluorescense microscopy or confocal steady state microscopy, photo-activate laser microscopy (PALM), time resolved anisotropic imaging microscopy (TRAIM) or a combination of two or more of these techniques. Emission, as used herein, is a synonym for luminescence. Accordingly, emission includes fluorescence and phosphorescence. Emission techniques can rely on all or a combination of these.

The technique may be used to observe emission lifetimes. The emission lifetimes may be observed over a period of at least 10 nano-seconds, more preferably 100 nano-seconds, even more preferably 1 microsecond and most preferably up to 1000 microseconds.

The technique according to the present invention may be used to image and/or map live cells and/or to label DNA and/or RNA in situ. It should be understood that in situ refers to work carried out inside or outside living cells and on other samples (such as free RNA) as disclosed herein. The types of imaging includes steady-state and time-resolved imaging.

The present invention may further comprise:

1) adding the complex as herein described to a cell;

2) optionally incubating the cell; and

3) performing an imaging step to locate the complex in the cell.

The step of adding the complex to the cell may involve attaching the complex to a chemical species.

The step of adding the complex to the cell may comprise the step of allowing the complex to diffuse into the cell.

The optional incubation step may occur for a period of from 1 and 30 minutes, more preferably from 2 and 20 and most preferably about 5 minutes.

The present invention further provides a transition metal complex having a tridentate ligand, which complex is bound to a biomolecule. The complex is preferably bound to the biomolecule via the ligand.

The biomolecule may comprise, for example, a protein, antigen, virus, DNA, RNA, or an antibody.

The present invention further provides for the use of a transition metal complex having a tridentate ligand as a labelling agent.

In one preferred aspect, the present invention relies on highly emissive, synthetically versatile and photostable platinum(II) complexes that make TREM a practicable reality.

In particular, the inventors have found that [PtLCl] complexes, {HL=1,3-di(2-pyridyl)benzene and derivatives}, are charge-neutral, small molecules which have low cytotoxicity and accumulate intracellularly within a remarkably short incubation time of five minutes, apparently under diffusion control. Their microsecond lifetimes and emission quantum yields of up to 70% are exceptionally high for transition metal complexes and has permitted the application of TREM to be demonstrated in a range of live cell types, for example normal Human Dermal Fibroblast, neoplastic C8161 human melanoma cells and Chinese Hamster Ovary cells. [PtLCl] are suitable emission labels for any eukaryotic cell types.

The high photostability of [PtLCl] under intense prolonged irradiation has allowed the first instance of tissue-friendly NIR two-photon excitation (TPE) in conjunction with transition metal complexes in live cells. A combination of confocal one-photon excitation, non-linear two-photon excitation, and microsecond time-resolved imaging has revealed: i) preferential localisation of the complexes to intracellular nucleic acid structures, in particular the nucleoli, and ii) the possibility of measuring intracellular emission lifetimes in the microsecond range. The combination of TREM, TPE and Pt(II) complexes is therefore now a powerful tool for investigating intracellular processes in vivo and/or in vitro, since the long lifetimes allow discrimination from autofluorescence.

The inventors' work introduces a new and powerful combination of highly emissive charge-neutral platinum(II) complexes with time-resolved imaging in TREM. The Pt(II) complexes developed also meet other essential criteria for TREM agents, including chemical and photochemical stability, low cytotoxicity, and synthetic versatility for potential specific targeting. This is of direct importance to biological imaging if implemented in combination with two-photon excitation (TPE). TPE is emerging as a versatile tool for non-invasive imaging of live cells and tissues. It excites chromophores in the visible region using simultaneous absorption of two photons of low-energy light in the range 600-1100 nm of relative tissue transparency. This enables z axis imaging depths of hundreds of micrometers. The high photon flux used in TPE provides intrinsic spatial resolution but demands exceptional photostability of the chromophores employed. The Pt(II) complexes used in the present invention have sufficient photostability for multiphoton excitation in live cells.

The present invention enables time-resolved emission imaging microscopy to be a practicable reality by using highly emissive, photochemically robust transition metal complexes for live cell imaging and mapping.

The present invention will now be described further with reference to the following non-limiting examples and figures, provided by way of example, in which:

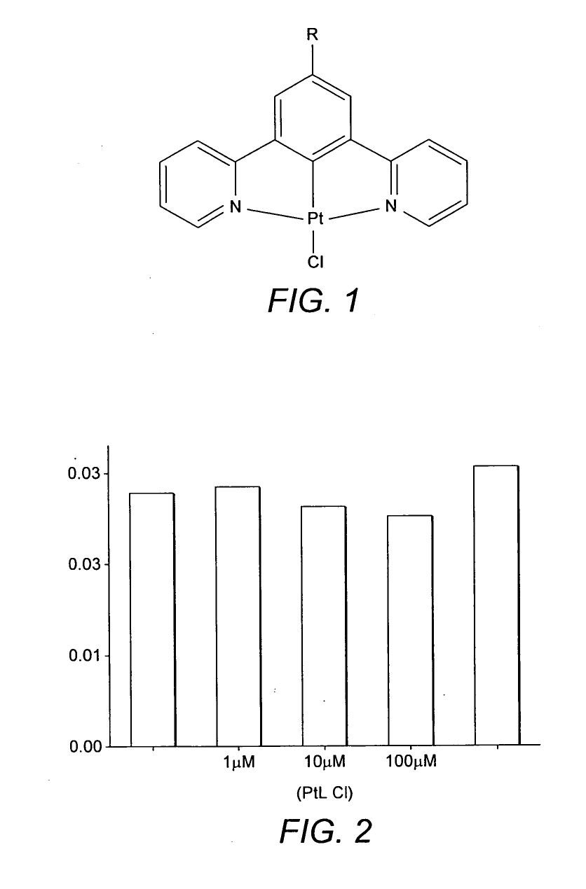

FIG. 1

Structure of the complexes [PtLⁿCl]: for n=1-4, R═H, —C(O)OCH₃, —CH₃, —C₆H₄—N(CH₃)₂, respectively.

FIG. 2

CHO K1 cell viability determined by MTT (OD). Cells were incubated for 5 minutes with the [PtL¹Cl] at different concentrations, washed with PBS (×3) and then incubated with fresh culture medium for 1 h. No significant difference between test and control conditions observed (mean±SEM, n=3).

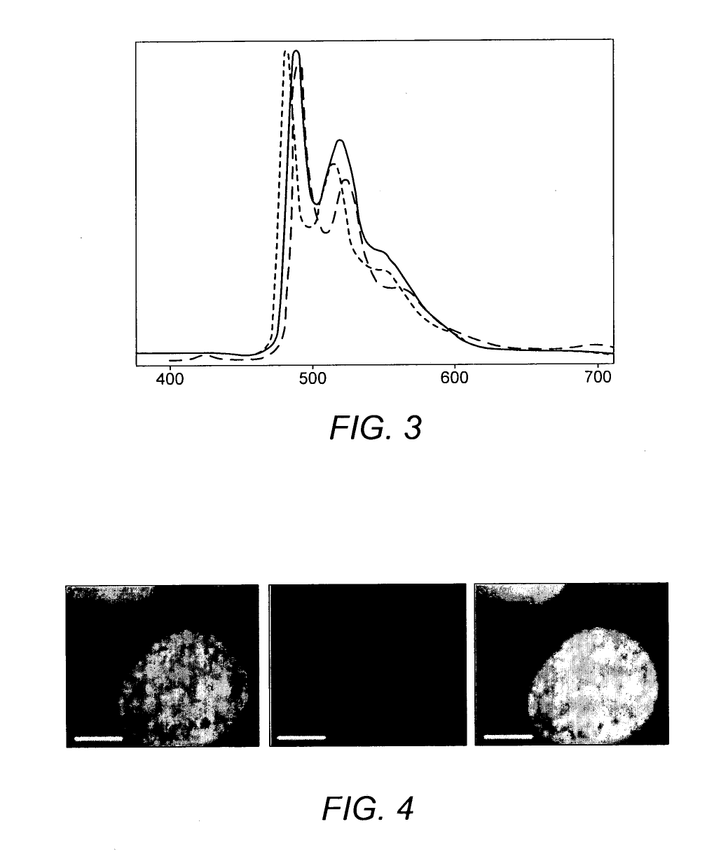

FIG. 3

The emission spectrum of [PtL¹Cl] in water (—), in aerated CH₂Cl₂ solution (—) and that obtained from the nucleus of HDF cells (solid line). The Emission Intensity (AU) is shown on the y axis and wavelength in nm is shown on the x axis.

FIG. 4

Determination of intracellular localisation by co-staining experiments. CHO-K1 cells in culture were incubated for 5 minutes with 100 μM of [PtL¹Cl] and fixed using 4% (w/v) paraformaldehyde. Cell nuclei were dual labelled using DAPI (at 300 nM). Samples were mounted for epifluorescence microscopy and visualised using a 100× oil immersion objective lens. a) [PtL¹Cl] localisation was identified using the FITC channel (λₑₓ=485 nm; λₑₘ=520 nm). b) DAPI was used to identify nuclei (λₑₓ=400 nm; λₑₘ=460 nm). c) Superimposed FITC and DAPI images. The images shown are representative of three experiments performed. Bar=5 μm.

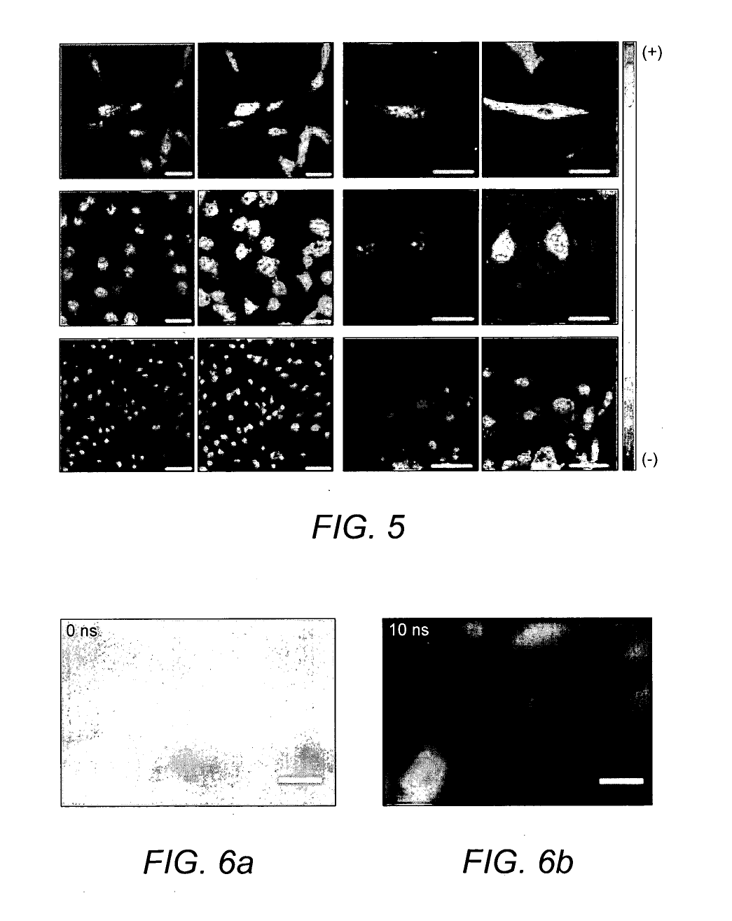

FIG. 5

Confocal micrographs of human dermal fibroblasts (top line), C8161 human melanoma (middle line) and Chinese hamster ovary cells (bottom line). Cells were incubated as in FIG. 4, washed with PBS (5 minutes) and imaged using an excitation wavelength of 488 nm. Paired images show greyscale (left) and rainbow (right) intensities respectively, identifying regions of [PtL¹Cl] accumulation. Z distances varied from 15 to 30 μm. Bar=10 μm. Relative intensities are shown on the scale on the right-hand-side with high (+) intensities at the top.

FIG. 6

Time-gated cellular imaging: live CHO cells pre-incubated with [PtL¹Cl], imaged in the presence of solution of fluorescein in 1M NaOH. The images were taken at 0 ns (a, left) and at 10 ns (b, right) delays after the 355 nm laser pulse. Bar=10 μm.

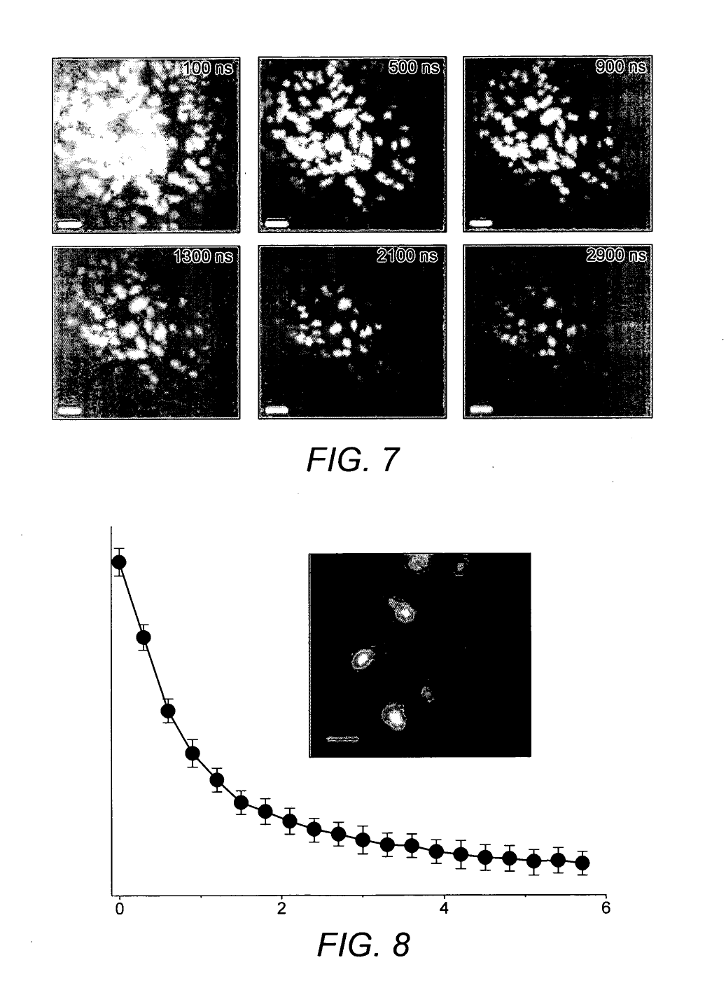

FIG. 7

Time-resolved gated emission images of live CHO cells incubated with [PtL¹Cl]. The images were recorded following 355 nm laser excitation at the time delays shown between 100 to 2900 ns from the laser flash. The time gate used was 100 ns, exposure time 0.02 s, 5 accumulations per time delay. Bar=50 μm.

FIG. 8

Emission kinetic trace obtained from the nuclei of live CHO cells (inset, bar=10 μm) incubated for 5 minutes with 100 μM solution of [PtL¹Cl]. Solid line represents a biexponential fit to the data. The data presented are averaged over at least three independent regions. The emission intensity is on the y axis. The time in μs is on the x axis.

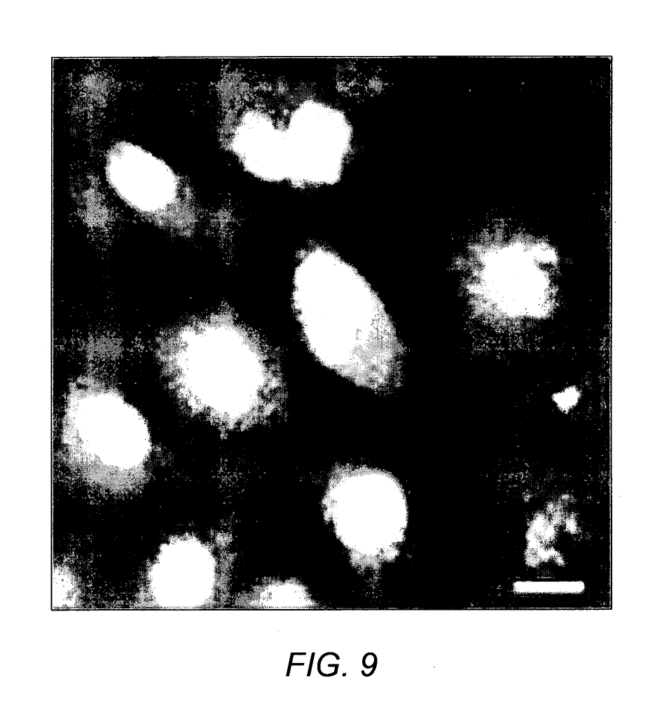

FIG. 9

A two-photon excitation high resolution emission image of live CHO cells incubated with [PtL¹Cl] obtained under 760 nm, 180 fs excitation. Bar=10 μm.

### EXAMPLES

1. Compounds Employed and Photophysical Background

The imaging agents used are [PtLCl] (FIG. 1), the Pt(II) complexes of the cyclometalating, terdentate, N̂ĈN-coordinating ligand 1,3-di(2-pyridyl)benzene and derivatives. These compounds can be readily synthesized in two steps from simple starting materials in high yields (see for example Williams J A G, Beeby A, Davies E S, Weinstein J A, Wilson C (2003) An alternative route to highly luminescent platinum(II) complexes: cyclometalation with N̂ĈN-coordinating dipyridylbenzene ligands. Inorg Chem 42:8609-8611). The core structure is easily derivatized at the central position of the ligand (R, FIG. 1) which allows for tuning of the emission over a wide spectral range from blue-green to orange. It also offers a means for simple conjugation to bio-targeting functionality; for example, biotin appended complexes have been prepared via an amide linker, R═CH₂NHCO. The straightforward synthetic route makes this class of labels unprecedentedly accessible, especially when compared with organic phosphorescent probes. [PtLCl] absorbs strongly in the UV region (350-380 nm, ε˜10⁴ mol⁻¹ dm³ cm⁻¹, with a weaker S→T band in the visible around 490 nm, ε˜200 mol⁻¹ dm³ cm⁻¹) and is intensely luminescent in fluid solution at room temperature, in the range 480-600 nm. The quantum yields of emission (Φₗᵤₘ) are 0.6-0.7 in degassed dichloromethane solution, remarkably high for platinum chromophores, in which the emissive excited state is frequently quenched by non-radiative decay via a low-lying, strongly antibonding d-d excited state. The high efficiency of luminescence can be rationalized in terms of the very strong ligand field associated with the rigid terdentate cyclometalating ligand, which raises the energy of d-d excited states, thereby diminishing or even eliminating this pathway of non-radiative decay. The emission of [PtLCl] can be attributed to an excited state of predominant triplet intra-ligand π-π* character, in which there is sufficient contribution from the metal to achieve a lifetime of several microseconds. Thus, from the photophysical perspective, the complexes satisfy the requirements for time-resolved imaging on the timescale of hundreds of nanoseconds to microseconds. The sections that follow discuss investigations into their intracellular applicability. These examples focus on the simplest [PtL¹Cl] complex (R═H). Similar results have also been obtained for derivatives bearing a —C(O)OCH₃, —C₆H₄—N(CH₃)₂, or —CH₃ group at the 4′-position.

2. Loading of Cells In Vitro with [PtL¹Cl] Complex—Cytotoxicity, Cell Viability and Photostability

Three cell lines were selected on the basis that they spanned a range of phenotypes, viz. (i) a normal human cell type (dermal fibroblast; HDF) that can typically be cultured up to passages 8-9; (ii) a neoplastic human cell line that can be cultured indefinitely (melanoma; C8161); and (iii) an animal derived cell line that can be cultured indefinitely (Chinese Hamster Ovary; CHO). HDF cells were isolated from the dermis of a healthy patient undergoing elective surgery, C8161 cells were derived from a highly metastatic secondary invasive melanoma, and the CHO line is a commonplace animal cell type used in many laboratories for genetic transfection and protein expression studies. All cells were mononucleated and adherent, and experimentation undertaken on cells at all stages of the division cycle (G1, S, G2 and M-phase). This provided a basis for identifying general intracellular target labeling sites, through membrane permeability and relative biochemical affinity. The three cell types studied also enabled potential labeling discrepancies to be identified due to differences in neoplastic properties or species.

To determine whether [PtL¹Cl] was potentially cytotoxic or altered cell viability, the metabolic activity of each cell type was examined using an MTT assay following exposure to the complex {MTT=3-(4,5-dimethylthiazol-2-yl)-2,5-diphenyltetrazolium bromide}. Concentrations of [PtL¹Cl] in the range from 1 to 100 μM were investigated with incubation times varying from 5 minutes to 24 hours. These studies revealed that the cells were viable after one hour of incubation with a 100 μM solution of [PtL¹Cl], with no significant decrease in metabolic activity (FIG. 2). Exposure to a 100 μM solution for 24 hours caused a 90% reduction in the viability of CHO cells, and a 70% reduction in the viability of HDF cells. However, exposure to a 10 μM solution did not cause significant reduction in cell viability even over a period of 24 hours.

The low cytotoxicity of the complexes within the cells is surprising given that, in fluid organic solvents, [PtL¹Cl] is a moderately efficient sensitiser of singlet oxygen (quantum yield for ¹Δ_{g} O₂ formation is 0.4). This suggests that, within the cell, the complex is protected from diffusion-controlled quenching by oxygen, possibly by binding to hydrophobic environments of large biomolecules such as proteins or nucleic acids—a notion supported by the results of co-staining studies, high resolution images, and spectroscopic measurements described below. No photobleaching of the compound within the cells was observed under prolonged NIR (780 nm) or UV (390 nm) laser irradiation at an average power of 100 mW for 6 hours.

3. Epifluorescence Microscopy and Steady-State Spectroscopy

Cells were incubated for 5 minutes in a 100 μM solution of [PtL¹Cl] in phosphate buffered saline (PBS) solution, containing 1% DMSO (v/v) to facilitate solubilisation of the complex. Control experiments confirmed that this concentration of DMSO had no adverse effect on cell viability as measured by MTT assay (FIG. 2). After incubation, the solution containing the complex was removed, the live cells were washed with PBS, and transferred on a cover slip to the microscope stage for direct imaging without being fixed. An incubation time of only 5 minutes was sufficient to achieve maximal emission intensity of [PtLCl] in the cells, which is strikingly shorter than that for the previously explored cationic metal complexes. The exceptionally short incubation time suggests that surprisingly [PtL¹Cl] is highly permeable across the plasma membrane of all three cell types studied and accumulates intracellularly under diffusion control. It is highly likely that no specific trans-membrane transport mechanism is required, via membrane channels or receptor mediated endocytosis, and demonstrates the potential of these Pt(II) compounds for use as labels in a wide range of eukaryotic cell types.

The Pt(II) complex in the cells emits a characteristic green-to-yellow light upon excitation. A typical spectrum of emitted light from stained CHO, HDF and C8161 cell nuclei is essentially identical in profile to that of [PtL¹Cl] in aqueous solution (FIG. 3), with the emission maxima in the range 490-550 nm. The characteristic vibrational structure of the emission spectrum, with the 0-0 band of the vibrational progression being the most intense, reflects the low degree of reorganization in the emissive excited state compared to the ground state. That a similar spectrum is obtained for the complex inside the cells to that in solution confirms convincingly that the “PtL” unit responsible for emission is retained intact within the cells.

An apparent preferential labelling of the nuclei was investigated by co-staining experiments with the standard nuclear stain DAPI (4′,6-diamidino-2-phenylindole), performed by epifluorescence microscopy. Conveniently, the emission spectrum of [PtL¹Cl] does not overlap with that of DAPI, and the former can be excited into the S→T absorption band centred at 488 nm where DAPI does not absorb, allowing for an independent visualisation of the localisation of both compounds within the cells. CHO or HDF cells were pre-incubated with [PtL¹Cl] and fixed; this was followed by staining with 300 nM DAPI for 5 minutes. FIG. 4a shows [PtL¹Cl] localisation in CHO cells visualized by its characteristic green emission; the localisation of DAPI (FIG. 4b) was visualised by its blue fluorescence at 460 nm under 400 nm excitation. The super-imposed [PtL¹Cl] and DAPI images (FIG. 4c) confirm the preferential accumulation of [PtL¹Cl] in the nucleus.

4. Live Confocal Steady-State Cell Imaging Under One-Photon Excitation

To investigate further the sub-cellular localisation of the [PtL¹Cl] chromophore, 15 to 30 orthogonal confocal 1 μm z-images were taken for each cell type using the above loading conditions. Representative images from each are shown in FIG. 5, again confirming preferential labelling of the nuclei. Well-defined regions of high intensity emission were identified corresponding to nucleoli sub-organelles (vide infra). Intensity plots were calibrated for each of the cell images confirming predominant nucleoli localisation, and are illustrated next to the [PtL¹Cl] greyscale micrographs (FIG. 5). Recent studies of nucleoli revealed structural organisation around ribosomal repeat DNA gene clusters, which give rise to 28S, 18S and 5.8S ribosomal RNAs. In turn, these are processed and assembled into ribosomes. It is also known that during the cell cycle, nucleoli can exist either individually or in clusters. Confocal images of CHO, fibroblast and C8161 cells identified cluster structures within the nuclei of all cells studied (FIG. 5). Confocal image observations made are therefore consistent with intracellular binding to DNA and, due to the heterogeneous composition of the nucleoli, would also suggest that [PtL¹Cl] may bind to RNA. The observed weak staining of the cytoplasm was not uniform. It was noted that the areas of brighter staining in the cytoplasm resembled structures likely to correspond to mitochondria, which would therefore also contain DNA. Interaction of [PtL¹Cl] with nucleic acids was confirmed by solution titrations with calf thymus and salmon sperm DNA in aqueous phosphate or HEPES buffer in the presence of NaCl. Emission of [PtL¹Cl] was enhanced in the presence of nucleic acid, with a saturation ratio of approximately 1:2.3 of the complex:DNA base pairs.

Binding to ribonucleic acids is also possible in view of the observed presence of [PtL¹Cl] in the cytoplasm and preferential nuclear regions. Such displacement of the chloride of [PtL¹Cl] by the N-heterocyclic donors of nucleobases as a mechanism of binding is not ruled out by the emission data, since emission originates from the “Pt-L” fragment. Notably, the aqua and pyridyl adducts [PtL(H₂O)]⁺ and [PtL(py)]⁺, have very similar spectra to that of [PtLCl].

Finally, no diffusion of [PtL¹Cl] out of the cells was observed over a period of several hours, which is also indicative of strong binding of the luminophore to subcellular structures.

5. Time-Resolved Imaging

Given its high emission intensity within the cell, and the long luminescence lifetime in vitro, [PtL¹Cl] is potentially an excellent and unprecedented candidate for time-resolved imaging on the microsecond timescale.

Lifetime imaging experiments on the live cells stained with [PtL¹Cl] were performed using pulsed laser excitation at 355 nm of pulse length ˜0.6 ns. Time resolved images were obtained with a time-gated CCD camera, which allows a series of images to be recorded at different time delays after the excitation pulse. A simple yet striking example of the power of time-gating is demonstrated by low-resolution images obtained from cells pre-treated with [PtL¹Cl] in the presence of fluorescein dianion {Γ_{f}=3.6 ns}, whose emission serves as a challenging model of short-lived background fluorescence. Immediately after the laser excitation pulse (FIG. 6a), little can be resolved because the fluorescein emission swamps the image. By activating the camera after a delay of 10 ns following the laser excitation pulse, the cells are visualized by the long-lived emission from the Pt(II) complex (FIG. 6b). These experiments clearly show the possibility and power of imaging with [PtL¹Cl] without interference from the autofluorescence background.

A representative set of time-resolved images obtained from the CHO cells pre-incubated with [PtL¹Cl] within the time frame from 50 to 2900 ns after the laser pulse is shown in FIG. 7. Remarkably, even 3 μs after the excitation laser pulse, the image is still of sufficient contrast for the cells to be visualized.

The quantitative kinetics of the decay of the emission intensity could be readily monitored in different cells and within different regions within the same cell (FIG. 8). The observed temporal decay in each case fits well to a biexponential function, where the major component has a lifetime of 760±100 ns. This compares with a value of 580±±30 ns measured for [PtL¹Cl] in air-equilibrated aqueous solution at room temperature. The somewhat longer lifetime in the cells probably reflects a level of protection from diffusion quenching by oxygen upon binding to large molecules such as nucleic acids which, as discussed earlier, may in turn account for the lack of apparent singlet-oxygen-induced cytotoxicity.

That the emission does not follow the single exponential decay observed in solution is clearly a reflection of the disparate-local environments and binding modes anticipated for the complex within the cell organelles. The observed biexponential fit is probably a simplest approximation to polyexponential decay kinetics arising from a distribution of environments, consistent with several subcellular structures being potential binding sites. These data highlight the ease with which the kinetic profile can be generated on the timescale of several microseconds in TREM.

6. Live Cell Imaging Under Multi-Photon Excitation

Another objective of the present invention, apart from the time-resolution, is multi-photon excitation of these bright and photostable Pt(II) luminophores within live cells. In particular, two-photon excitation. This brings into play the benefits of TPE, most importantly the intrinsic high resolution, and ability to excite labelling agents at low energies in the near-IR.

7. Determination of the Two-Photon Absorption Cross-Section of [PtLCl]

The two-photon absorption cross section, δ, of the Pt(II) complexes was determined by monitoring the emission intensity in DMF solution under 790 nm excitation, using fluorescein and rhodamine-B as standards. The value of 4 (±2)×10⁻⁵⁰ cm⁴ s/photon (4 GM) obtained is lower than that of fluorescein but easily sufficient for practical applications. The two-photon nature of the excitation process was confirmed by the quadratic increase of the intensity of emitted light with the increase in laser power. The emission lifetime measured following excitation at 790 nm was identical to that observed under one-photon excitation at 355 nm, confirming that the same excited state was formed in both cases.

8. High Resolution Confocal Imaging Under Two-Photon Excitation

Two photon excitation of [PtL¹Cl] in live CHO cells was accomplished using a mode-locked Ti-sapphire laser operating at 758 nm. High resolution images of the cells were generated by rastor scanning of the laser spot in the xy plane (FIG. 9). The images clearly confirm the preferential localisation of the chromophore in the nucleoli that had been observed in the linear confocal experiments described above. The results demonstrate the viability of TPE of the platinum complexes in live cells, with apparently no short-term detrimental effect on the cells. This is believed to be the first example of two-photon imaging in live cells using transition metal complexes.

9. Materials and Methods

The platinum(II) complexes were prepared as described previously. The details of cell culture sources and handling are conventional.

10. Confocal and Epifluorescent Imaging Under One-Photon Excitation and Co-Staining

A Zeiss LSM 510 confocal microscope with 20× and 40× long-range water-dipping lenses was used. [PtLCl] in cells were excited with an Ar-ion laser at 488 nm, emission was monitored through a 505-530 nm filter reflected from a 545 nm dichroic mirror. Image data acquisition and analysis were carried out with Carl Zeiss Laser Scanning Systems LSM 510 software, version 3.2 (Carl Zeiss GmbH, Germany). Epifluoresecnce microscopy was carried out using a Leica-DM-IRB inverted microscope using epifluorescent illumination and a 100× oil immersion lens at 485 nm and acquisition at 520 nm for [PtLCl], and illumination at 400 nm and acquisition at 460 nm for DAPI labelled nuclei.

Two-photon cross-sections were measured in 40 μM DMF solution as described by Xu and Webb using an 8 W 532 nm (Verdi, Coherent Ltd) pumped Mira 900-F Ti-sapphire laser producing 180 fs pulses at 790 nm using fluorescein as a standard (δ=38±9.7 GM). The laser light was focused onto the stage of an inverted microscope (Nikon TE2000U) using a ×40, NA 0.9 microscope objective. The fluorescence was collected by the same objective, through a dichroic mirror (660IK, Comar) and imaged onto a fast micro channel plate photo multiplier tube (PMT). The signal from the PMT was synchronised to the laser pulses using a Becker-Hickl SPC700 time correlated single photon counting module. Relative absorption cross-section (δ) values of rhodamine B and fluorescein were first determined to ensure that the experimental set-up yielded data consistent with those previously reported in the literature. The uncertainty in the δ value arises from the usual difficulties of calibration in such measurements, augmented by the number of corrections required for the effects of oxygen and intermolecular self-quenching on Φₗᵤₘ.

11. Time-Resolved Imaging Experiments

The actively Q-switched nanosecond AOT-YVO-20QSP/MOPA Nd:vanadate diode pumped microlaser with repetition rate (1-20 kHz), short pulse duration (0.6 ns), 355 nm (12 mJ/pulse) was operated in the external trigger mode. Pulses generated from a Stanford DG535 pulse delay generator (PDG) were used as the trigger signals for the laser. The 355 nm laser pulses were directed to the epifluorescence port of the microscope (Nikon, TE200U) and reflected off a dichroic mirror (410BK, Comar) and into the back aperture of a ×40 lens (NA 0.9). Phosphorescence from the sample was collected by the same objective and imaged onto a sub-nanosecond gated intensified CCD (Andor iStar) with delays set by the PDG and synchronized to the nanosecond laser. The laser light was blocked using a 420GY (Comar) filter in front of the CCD camera. The three exit ports of the microscope were utilized to direct the output to the gated CCD camera, a steady-state Q-Cam 10-bit colour camera, or an iDus CCD camera, depending on the particular observation mode. For the gated CCD, typical exposure time was 0.02 s, the number of accumulations varied from 5 to 20. The principle of time-gating is demonstrated in FIG. 6. By monitoring “time slices” at increasing intervals after the pulse, e.g., every 100 ns, the intensity of the image can be monitored as a function of time.

Imaging under two photon excitation was performed using the Ti-sapphire laser and inverted microscope described above. Images were generated point-by-point by raster scanning of the laser spot using an x,y galvanometer (GSI Lumonics, USA), fluorescence was detected using the Hamamatsu PMT (R3809U) operating in single photon counting mode.

The present invention allows the microsecond domain in imaging, including that of biological structures, and is particularly suited to time-resolved emission imaging microscopy (TREM). The previous obstacles to practical TREM have been overcome by the identification of brightly emissive, photostable and low-cytotoxic Pt(II) complexes as imaging agents for live cells cultured in vitro or in vivo. The exceptionally high emission quantum yields and appropriately long lifetimes of these chromophores allowed for gated emission experiments and time-resolved lifetime-based imaging on a hitherto uncharted timescale. These small, charge-neutral compounds are remarkable in their simplicity and in the ease with which they can be synthesized, and derivatized should specific targeting be required.

Maximum emission intensity within all cell types studied, foe example CHO, HDF and melanoma C8161, was achieved within a strikingly short incubation period of five minutes, indicating that no specific trans-membrane transport mechanism is required and that the compounds are amenable to use as labels in any eukaryotic cell types. The photostability of [PtL¹Cl] under prolonged intense irradiation allowed, for the first time, the use of NIR two-photon excitation in conjunction with transition metal complexes in live cells. The high-resolution two-photon images confirmed the preferential accumulation of the Pt(II) complex in the nuclei, and in particular in the nucleoli. The intracellular preferential target site proposed for [PtL¹Cl] is DNA, which may extend to RNA.

The present invention shows the power of lifetime mapping and two-photon excitation for imaging of live cells and provides an approach for non-invasive imaging of many hundreds of micrometers into complex biological structures. Extension to antibody conjugation, where high fluorophore photostability is essential, is also possible.

### BACKGROUND REFERENCES TO THE PRESENT INVENTION

Johnsson N, Johnsson K (2007) Chemical tools for biomolecular imaging. ACS Chem Biot 2:31-38.
Bachmann L, Zezell D M, Ribeiro A D, Gomes L, Ito, A S (2006) Fluorescence spectroscopy of biological tissues—a review. Appl Spec Rev 41:575-590.
Tsien R Y (1992) in Fluorescent chemosensors for ion and molecule recognition, ed. Czarnik AW (American Chemical Society, Washington D.C.), pp 130-146.
De Silva A P, et al (1997) Signaling recognition events with fluorescent sensors and switches. Chem Rev 97:1515-1566.
Lakowicz J R (2006) Principles of Fluorescence Spectroscopy (Springer, New York), pp 741-755; Botchway S W, Parker A W, Bisby R H, Crisostomo A G (2008) Real-time cellular uptake of serotonin using fluorescence lifetime imaging with two-photon excitation. Microscopy Research and Technique, 71: 267-273.
Suhling K, French P M W, Phillips D (2005) Time-resolved fluorescence microscopy. Photochem Photobiol Sci 4:13-22.
Chen Y, Barkley M D (1998) Toward understanding tryptophan fluorescence in proteins. Biochemistry 37:9976-9982.
Treanor, B., et al (2005) Imaging fluorescence lifetime heterogeneity applied to GFP-tagged MHC protein at an immunological synapse. J Microscopy—Oxford 217, 36-43.
Beeby A, et al (2000) Luminescence imaging microscopy and lifetime mapping using kinetically stable lanthanide(III) complexes. J Photochem Photobiol B 57:83-89.
Puckett C A, Barton J K (2007) Methods to explore cellular uptake of ruthenium complexes. J Am Chem Soc 129:46-47.
Amoroso A J, et al (2007) Rhenium fac tricarbonyl bisimine complexes: biologically useful fluorochromes for cell imaging applications. Chem Commun 3066-3068.
Lo K K W, Louie M W, Sze K S, Lau J S Y (2008) Rhenium(I) polypyridine biotin isothiocyanate complexes as the first luminescent biotinylation reagents: synthesis, photophysical properties, biological labeling, cytotoxicity, and imaging studies. Inorg Chem 47:602-611.
de Haas R R, et al (1997) Platinum porphyrins as phosphorescent label for time-resolved microscopy. J Histochem Cytochem 45:1279-1292.
de Haas R R, et al (1999) Phosphorescent platinum/palladium coproporphyrins for time-resolved luminescence microscopy. J Histochem Cytochem 47:183-196.
Siitari H, Hemmila I, Soini E, Lovgren T, Koistinen V (1983) Detection of hepatitis-B surface-antigen using time-resolved fluoroimmunoassay. Nature 301:258-260.
Pandya S, Yu J, Parker D (2006) Engineering emissive europium and terbium complexes for molecular imaging and sensing. Dalton Trans 2757-2766; Vereb G, Jares-Erijman E, Selvin P R, Jovin T M (1998) Temporally and spectrally resolved imaging microscopy of lanthanide chelates. Biophys J. 74:2210-2222.
Gabourdes M, Bourgine V, Mathis G, Bazin H, Alpha-Bazin B (2004) A homogeneous time-resolved fluorescence detection of telomerase activity. Anal Biochem 333: 105-113.
Williams J A G, Beeby A, Davies E S, Weinstein J A, Wilson C (2003) An alternative route to highly luminescent platinum(II) complexes: cyclometalation with N̂ĈN-coordinating dipyridylbenzene ligands. Inorg Chem 42:8609-8611.
Farley S J, Rochester D L, Thompson A L, Howard J A K, Williams J A G (2005) Controlling emission energy, self-quenching and excimer formation in highly luminescent N̂ĈN-coordinated platinum(II) complexes. Inorg Chem 44:9690-9703.
McMillin D R, Moore J J (2002) Luminescence that lasts from Pt(trpy)Cl⁺ derivatives (trpy=2,2′:6′,2″-terpyridine). Coord Chem Rev 229:113-121.
Williams J A G (2007) Photochemistry and photophysics of coordination compounds: platinum. Topics Curr Chem 281:205-268.
Boisvert F M, van Koningsbruggen S, Navascues J, Lamond A I (2007) The multifunctional nucleolus Nature Rev Mol Cell Biol 8:574-585.
Lippard S J (1978) Platinum complexes: probes of polynucleotide structure and antitumor drugs. Acc Chem Res 11:211-217.
Peyratout C S, Aldridge T K, Crites D K, McMillin D R (1995) DNA-binding studies of a bifunctional platinum complex that is a luminescent intercalator. Inorg Chem 34:4484-4489.
Hofmann A, Dahlenburg L, van Eldik R (2003) Cyclometalated analogues of platinum terpyridine complexes: kinetic study of the strong □-donor cis and trans effects of carbon in the presence of a □-acceptor ligand backbone. Inorg Chem 43:6528-6538.
Griffith O W (1999) Biologic and pharmacologic regulation of mammalian glutathione synthesis. Free Rad Biol Med 27: 922-935.
Tarran W A, M. Chem. Thesis, University of Durham, 2005. Lessing H E, Richardt D, von Jena A, (1982) Quantitative triplet photophysics by picosecond photometry. J Mol Struct 84:281-292.
Xu C, Webb W W (1996) Meausurement of two-photon excitation cross sections of molecular fluorophores with data from 690 to 1050 nm. J Opt Soc Am B 13:481-491.

1. The use of a transition metal complex having a tridentate ligand in an imaging technique.

2. Use as claimed in claim 1, wherein the transition metal complex is used as a labelling agent.

3. Use as claimed in claim 2, wherein the transition metal complex is used as a labelling agent in a cell.

4. Use as claimed in claim 1, wherein the transition metal complex is introduced into a cell in vitro or in vivo.

5. Use as claimed in claim 1, wherein the transition metal complex is pre-bound to a chemical species that is introduced into a cell.

6. Use as claimed in claim 5, wherein the chemical species is a protein, an antibody, DNA, RNA, an antigen or a virus.

7. Use as claimed in claim 1, wherein the transition metal complex binds to active sites within a cell to label at least a portion of the cellular structure.

8. Use as claimed in claim 7, wherein the active sites are nucleic acid active sites within a cell, preferably RNA and/or DNA active sites.

9. Use as claimed in claim 7, wherein the active sites are within the nucleus or nucleoli of a cell.

10. Use as claimed in claim 1, wherein the transition metal complex has a quantum yield of fluorescence emission of 0.6 or greater, more preferably 0.65 or greater and most preferably 0.7 or greater.

11. Use as claimed in claim 1, wherein the transition metal has a square planar coordination.

12. Use as claimed in claim 1, wherein the transition metal is platinum.

13. Use as claimed in claim 12, wherein the platinum complex is a Pt^{(II)} complex.

14. Use as claimed in claim 12, wherein the platinum complex is charge neutral.

15. Use as claimed in claim 12, wherein the platinum complex is of the formula Pt[L]X, wherein L is a tridentate ligand and X is a monodentate ligand.

16. Use as claimed in claim 12, wherein the tridentate ligand (L) is a cyclometallating ligand.

17. Use as claimed in claim 15, wherein the tridentate ligand (L) coordinates to the transition metal via N̂ĈN coordination points.

18. Use as claimed in claim 15, wherein the tridentate ligand (L) is 1,3-di(2-pyridyl)benzene or a derivative thereof.

19. Use as claimed in claim 18, wherein the tridentate ligand (L) is a 1,3-di(2-pyridyl)benzene derivative substituted at the 4′ position.

20. Use as claimed in claim 15, wherein the tridentate ligand (L) is substituted, preferably at the 4′ position, with a bio-targeting functionality or a linking group suitable for reactively attaching the derivative to a bio-targeting functionality.

21. Use as claimed in claim 20, wherein the linking group is an amide group or an ester group.

22. Use as claimed in claim 15, wherein X is a monodentate pi donor ligand.

23. Use as claimed in claim 15, wherein the transition metal complex has the formula: 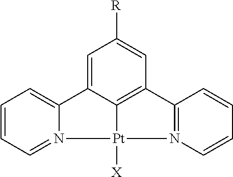
wherein R is —H, —C(O)OCH₃, —CH₃, or —C₆H₄—N(CH₃)₂; and
wherein X is a monodentate ligand.

24. Use as claimed in claim 23, wherein X is Cl, Br, F or OH.

25. Use as claimed in claim 24, wherein X is Cl.

26. Use as claimed in claim 1, wherein the imaging technique comprises microscopy.

27. Use as claimed in claim 1, wherein the imaging technique comprises photon imaging.

28. Use as claimed in claim 1, wherein the imaging technique comprises fluorescence microscopy.

29. Use as claimed in claim 1, wherein the imaging technique comprises fluorescence lifetime imaging microscopy (FLIM), time-resolved emission imaging microscopy (TREM), multi-photon excitation (MPE), two-photon excitation microscopy (TPE), Förster resonance energy transfer microscopy (FRET), epi-fluorescense microscopy or confocal steady state microscopy, photo-activate laser microscopy (PALM), time resolved anisotropic imaging microscopy (TRAIM) or a combination of two or more of these techniques.

30. Use as claimed in claim 1, wherein the technique is used to observe emission lifetimes.

31. Use as claimed in claim 30, wherein the emission lifetimes are observed over a period of at least 100 nano-seconds, more preferably 1 microsecond and most preferably up to 1000 microseconds.

32. Use as claimed in claim 1, wherein the technique is used to image and/or map live cells and/or to label RNA in situ.

33. Use as claimed in claim 1 and further comprising:
1) adding the complex to a cell;
2) optionally incubating the cell; and
3) performing an imaging step to locate the complex in the cell.

34. Use as claimed in claim 33, wherein before the step of adding the complex to a cell, the complex is attached to a chemical species.

35. Use as claimed in claim 33, wherein the step of adding the complex to a cell comprises a step of allowing the complex to diffuse into the cell.

36. Use as claimed in claim 33, wherein the optional incubation step occurs for a period of from 1 and 30 minutes, more preferably from 2 and 20 and most preferably about 5 minutes.

37. A transition metal complex having a tridentate ligand, which complex is bound to a biomolecule.

38. A transition metal complex as claimed in claim 37, wherein the complex is bound to a biomolecule via the ligand.

39. A transition metal complex as claimed in claim 37, wherein the biomolecule is a protein, antigen, virus, DNA, RNA, or an antibody.

40. The use of a transition metal complex having a tridentate ligand as a labelling agent.
